SEQUENCE LISTING (13 sequences)

SEQ ID NO: 1 - DNA, 798 bp, Oryza sativa
atggctcacgagatgatgggtggcttcttcggccatccgccgccgccgcctgcgacggcggcggtgggtgaggaggaggacgaggtggtggaggagacggaggagggtggccatggcggaggagttcaggggaagctttgcgcgaggggacactggcggccggcggaggacgccaagctcaaggacctcgtcgcgcagtacggcccccagaactggaacctcatcgccgagaagctcgacggcagatcagggaagagctgcaggctgaggtggttcaaccagctggacccgaggattaaccggagggcgttcacggaggaggaggaggagcggctcatggcggcgcaccgggcgtacggcaacaagtgggcgctcatcgcccgcctcttccccggccgcaccgacaacgccgtcaagaaccactggcacgtcctcatggcgcgccgccaccgcgagcagtccggcgccttccgccgccgcaagccttcctcctcctccgcctcccccgcccccgcccccgcgccgccgccgccgccgcagcccgtcgtcgcgctccaccaccaccaccaccggtactcgcagcagtacagcgggtacagcggcgccgccgagtccgacgagtcggcctccacctgcaccaccgacctctccctcagctccggcagcgccgcggccgccgccgccgccgccgcggcggcgaacatcccctgctgcttctaccagcgcggcggcgcccaagccaccaccgacagccacaccatccccttcttcgacttcctcggcgtcggcgcgacatga

SEQ ID NO: 2 - DNA, 1365 bp, Oryza sativa
catccatccattccagcatttcttgaagcttgctgtggatcagagaggatggctcacgagatgatgggtggcttcttcggccatccgccgccgccgcctgcgacggcggcggtgggtgaggaggaggacgaggtggtggaggagacggaggagggtggccatggcggaggagttcaggggaagctttgcgcgaggggacactggcggccggcggaggacgccaagctcaaggacctcgtcgcgcagtacggcccccagaactggaacctcatcgccgagaagctcgacggcagatcagggaagagctgcaggctgaggtggttcaaccagctggacccgaggattaaccggagggcgttcacggaggaggaggaggagcggctcatggcggcgcaccgggcgtacggcaacaagtgggcgctcatcgcccgcctcttccccggccgcaccgacaacgccgtcaagaaccactggcacgtcctcatggcgcgccgccaccgcgagcagtccggcgccttccgccgccgcaagccttcctcctcctccgcctcccccgcccccgcccccgcgccgccgccgccgccgcagcccgtcgtcgcgctccaccaccaccaccaccggtactcgcagcagtacagcgggtacagcggcgccgccgagtccgacgagtcggcctccacctgcaccaccgacctctccctcagctccggcagcgccgcggccgccgccgccgccgccgcggcggcgaacatcccctgctgcttctaccagcgcggcggcgcccaagccaccaccgacagccacaccatccccttcttcgacttcctcggcgtcggcgcgacatgattaccggcgacgtcctccggtggtggtggtgtacaggaaggaaggaaggaaggaagcagccaagatcaagcgatcaaaaccaagctaaggttagagttagccaagaacaagactaggacgaggaggaggaggaggaacaagacgtgctcgaagctcatcaatggcgttctcaaccaaacaagttttttctgctgctgctgctgctgcaagctagcttagattattcagatgaagaacaataagaagaagatgtagtactagtaaactagtaatggtgtgtgcgtttggaaatcaacaagcttccatgccatgccatgcctcctctgttcatcgatttcttttttttatctctctcctctgtatgaacaagtagaagaagaagaagaacaaattaatccattgctctcgcagatggatcagcaacatcaagcattgccgcattgtctctctctttgtgttcattgtccatgacgccaaagagaaaagaagaaaaattttcattgctctgttcttgaaatc

SEQ ID NO: 3 - DNA, 1665 bp, Oryza sativa
gatttcaagaacagagcaatgaaaatttttcttcttttctctttggcgtcatggacaatgaacacaaagagagagacaatgcggcaatgcttgatgttgctgatccatctgcgagagcaatggattaatttgttcttcttcttcttctacttgttcatacagaggagagagataaaaaaaagaaatcgatgaacagaggaggcatggcatggcatggaagcttgttgatttccaaacgcacacaccattactagtttactagtactacatcttcttcttattgttcttcatctgaataatctaagctagcttgcagcagcagcagcagcagaaaaaacttgtttggttgagaacgccattgatgagcttcgagcacgtcttgttcctcctcctcctcctcgtcctagtcttgttcttggctaactctaaccttagcttggttttgatcgcttgatcttggctgcttccttccttccttccttcctgtacaccaccaccaccggaggacgtcgccggtaatcatgtcgcgccgacgccgaggaagtcgaagaaggggatggtgtggctgtcggtggtggcttgggcgccgccgcgctggtcgacggcgccggggggctcgccgcggcccgccggggcggagaaggcggagcgcgcgctcggcgcgaacgtggacgccgccgcgggagccatggcgtgcggggcggcggcgaagtcgtagcctgcaccggggaagaacgccaccgtgtcagccgccgccgcgacgcgaggcgcgcggcacgcggcggtggaagaggaagaggcgcgcggcgtgctctggtagaagcagcaggggatgttcgccgccgcggcggcggcggcggcggccgcggcgctgccggagctgagggagaggtcggtggtgcaggtggaggccgactcgtcggactcggcggcgccgctgtacccgctgtactgctgcgagtaccggtggtggtggtggtggagcgcgacgacgggctgcggcggcggcggcggcgcgggggcgggggcgggggaggcggaggaggaggaaggcttgcggcggcggaaggcgccggactgctcgcggtggcggcgcgccatgaggacgtgccagtggttcttgacggcgttgtcggtgcggccggggaagaggcgggcgatgagcgcccacttgttgccgtacgcccggtgcgccgccatgagccgctcctcctcctcctccgtgaacgccctccggttaatcctcgggtccagctggttgaaccacctcagcctgcagctcttccctgccatgccatcccatcacaaacaccttcaatgcgcgtcgttcttgggcattgcacccaccgccatggaggagctcttacctgatctgccgtcgagcttctcggcgatgaggttccagttctgggggccgtactgcgcgacgaggtccttgagcttggcgtcctccgccggccgccagtgtcccctcgcgcaaagcttcccctgaactcctccgccatggccaccctcctccgtctcctccaccacctcgtcctcctcctcacccaccgccgccgtcgcaggcggcggcggcggatggccgaagaagccacccatcatctcgtgagccatcctctctgatccacagcaagcttcaagaaatgctggaatggatggatg

SEQ ID NO: 4 - protein, 265 aa, Oryza sativa
MAHEMMGGFFGHPPPPPATAAVGEEEDEVVEETEEGGHGGGVQGKLCARGHWRPAEDAKLKDLVAQYGPQNWNLIAEKLDGRSGKSCRLRWFNQLDPRINRRAFTEEEEERLMAAHRAYGNKWALIARLFPGRTDNAVKNHWHVLMARRHREQSGAFRRRKPSSSSASPAPAPAPPPPPQPVVALHHHHHRYSQQYSGYSGAAESDESASTCTTDLSLSSGSAAAAAAAAAAANIPCCFYQRGGAQATTDSHTIPFFDFLGVGAT

SEQ ID NO: 5 - DNA, 797 bp, Oryza sativa
atggctcacgagatgatgggtggcttcttcggccatccacgccgccgcctgcgacggcggcggtgggtgaggaggaggacgaggtggtggaggagacggaggagggtggccatggcggaggagttcaggggaagctttgcgcgaggggacactggcggccggcggaggacgccaagctcaaggacctcgtcgcgcagtacggcccccagaactggaacctcatcgccgagaagctcgacggcagatcagggaagagctgcaggctgaggtggttcaaccagctggacccgaggattaaccggagggcgttcacggaggaggaggaggagcggctcatggcggcgcaccgggcgtacggcaacaagtgggcgctcatcgcccgcctcttccccggccgcaccgacaacgccgtcaagaaccactggcacgtcctcatggcgcgccgccaccgcgagcagtccggcgccttccgccgccgcaagccttcctcctcctccgcctcccccgcccccgcccccgcgccgccgccgccgccgcagcccgtcgtcgcgctccaccaccaccaccaccggtactcgcagcagtacagcgggtacagcggcgccgccgagtccgacgagtcggcctccacctgcaccaccgacctctccctcagctccggcagcgccgcggccgccgccgccgccgccgcggcggcgaacatcccctgctgcttctaccagcgcggcggcgcccaagccaccaccgacagccacaccatccccttcttcgacttcctcggcgtcggcgcgacatga

SEQ ID NO: 6 - DNA, 33 bp, Oryza sativa
cgggatccgctatgcacctagacgagtgttgtc

SEQ ID NO: 7 - DNA, 33 bp, Oryza sativa
cgcacgtggtgaccactgagcaaggagtagctc

SEQ ID NO: 8 - DNA, 39 bp, Oryza sativa
tcggatccgagctctcttcgacttcctcggcgtcggcgc

SEQ ID NO: 9 - DNA, 40 bp, Oryza sativa
tcggtaccactagtcagaaaaaacttgtttggttgagaac

SEQ ID NO: 10 - DNA, 39 bp, Oryza sativa
tcactagtgagctctcttcgacttcctcggcgtcggcgc

SEQ ID NO: 11 - DNA, 42 bp, Oryza sativa
tcggatccggcgcgcccagaaaaaacttgtttggttgagaac

SEQ ID NO: 12 - DNA, 30 bp, Oryza sativa
tgccatggcctctctgatccacagcaagct

SEQ ID NO: 13 - DNA, 2199 bp, Oryza sativa
gctatgcacctagacgagtgttgtccacgtgcatagctagaaatacactatttagaacaaaggcagtatgaagaaaataaacaattaaaatatattttccaatcgtgttgtgtcaattatttgtacatgtcgttggccttttgtgagtgattactaaatcgataagaaaacaagtgaataataaataggccacgactattaccgtgttattgttttgattacatatctgccacatgaccatcctttgctagccctggtacaaaattggtattccatcgatgaattatccaactactcataacaattaaggaaaacatttagttacagttccaacctttacgagtagaacatgtccgatcaatacgctgactactctagttatttaaaatatattatccgctaggagacattaatatcttctttgtacgtacttgatgctgaccttcgggtatatgttaccgaatccactaaaagcctagtgaacgacaaatcacatccgttaccatattattgtttggatcatataattgtcgcacgattatcctttttggctctagtgcaaatttggtattatatagaccgtttcacctatctaacggcttacaatatttaagggacacatttagttacaatcgaaacccctacgagttatttaaagtatattatctgctcaagagcattaatgtcttgtttgttcgtactcgacgctaacctttgggggtacgttacagaatcgactaaaagccaagtaaacgacaaagccgcagccattaccgtattattgtttggatcacataaccgtcacacgattatccttgactggctctggtactaaattggcattatatggactgtttcacctatctaactaatcataatatcaagaaacatatttagtggcaattctaacctatacgagttgtttaaagtatattatccactcaagggcattaatgtctttgtttgtacgtattcaacgctgaccttcagggctacattacaaaatcgattgaaagcctatgaaacgacaaagccacgacagttaccgtattattgtttagatcacgtaaccgtcgtacgattatccttggctagatccggcacaaaattggcattaatatcgaccgtttcacttatccaaatagaaaagaaaacatttaagaaacacatgcagatatttttccaacctttccaactacaaccaatatgatcaacgagcggtgacactctctccaaacaaaaaccagcgacacttttgctcaaccggttagccctaaccaaagcgcataagtggtccccggtcctcgcgctagttttacccgtacaaatcttgactatacaaatcttctttgatcttcaaactaagattcatggccacatagagatgctaacaacaagatgaggaggagtgatggatggagttggaatgatgatagatgcaatgtggtctagtgtggtcacaatgaggaaaatgttgggtgtgaggatggtgtggggggtgcacatagggacaagggagttctggtgggtttgtcaaggctgcaagggacagagttccacccctgatagtggttgggttggagtcccaaacccacacgagcggcctctgaaacccaaaaaaaaaaaaagattcataaaaacaccccaagagaagaacagagatagaaatgaaaagaacacaagacaggaacaacaccatctctcttttccaacccttttgccatcgtcctcaaagtaagtgaaacccctccccacacaaaaaccaaatcctctcacatgaagataaaaaagaagaaccaccctaaccaaaccaaaacaattcagttcagagagagagagataagcacaatacagatagacataaatagagatatacacaaagattgttttttctttcaacaataaaaaaaactctacttttcttttaaggaggcctgtggcatttatgcatttctgttttgtttttgttggttgggagagtcgaagccaaagcccccctctcctctcttattcctatattagccccccctaccccattttgccgttcgctgcagaagcaacaacaccaacaaagctatagctgtctctttccttgcatctcctctcctcttctcttctcttatataggccaccgcgaggtcggtctccatccatccatccattccagcatttcttgaagcttgctgtggatcagagagg

(19) United States
(12) Patent Application Publication
(10) Pub. No.: US 2015/0106970 A1
(43) Pub. Date: Apr. 16, 2015

(54) BREEDING METHOD FOR TWO LINES HYBRID RICE BASED ON THE RICE OSMS4 GENE MUTANT

(71) Applicant: Dabing Zhang, Shanghai (CN)

(72) Inventor: Dabing Zhang, Shanghai (CN)

(73) Assignee: SHANGHAI JIAOTONG UNIVERSITY, Shanghai (CN)

(21) Appl. No.: 13/820,733

(22) PCT Filed: Nov. 29, 2010
(86) PCT No.: PCT/CN2010/079229

§ 371 Date: Mar. 4, 2013

(30) Foreign Application Priority Data
Jul. 27, 2010 (CN) 201010237721.6

(51) Int. Cl. C12N 15/01; C12N 15/82

(52) U.S. Cl. CPC C12N 15/01; C12N 15/8227; C12N 15/8225; C12N 15/8233; C12N 15/8231; C12N 15/8234
USPC 800/270; 536/24.1

(57) ABSTRACT

Provided is a breeding method for two lines hybrid rice based on the rice mutant osms4 (Oryza sativa male sterile 4), which was originally selected from the mutagenizing seeds of japonica cultivar 9522 with ⁶⁰Co gamma ray, and the stable inheritance osms4 mutant was obtained after three generations backcrossing, which is regulated by a recessive single gene OsMS4. The invention is related to the usage of OsMS4 gene (also known as Carbon Starved Anther, the CSA gene), osms4 mutant can be used as male sterile line under short day condition in both japonica and indica background, which grows abnormal anther with defection during second mitosis stage and produces completely sterile pollen. While under the long day condition, the osms4 mutant can be used as maintainer liner and produces normal pollen.

This application claims the priority of Application No. CN 201010237721.6, filed 27 Jul. 2010.

### BACKGROUND OF THE INVENTION

1. Technical Field

The present invention relates to a biotech application, and more particularly to a seed production, reproduction and breeding method for the two line hybrid rice based on the mutant of rice OsMS4 gene.

2. Description of Related Art

The male sterile line is the maternal rice with abnormal development or degeneration (degeneration of an anther or pollen) of a male reproductive organ but normal development of a female reproductive organ. As the abnormalities in the anther or the pollen lead to a non-pollen plant or inactive pollen, the rice is incapable of self-pollinating and setting seeds, so it is fertilized with foreign pollens and sets seeds. Therefore, with the maternal rice used as a genetic tool, the artificial supplementary pollination produces a large number of hybrid seeds. In terms of breeding strategies, there are three development stages for the hybrid rice, namely three line method, two line method and one line method. Each stage marks a breakthrough in breeding, thereby lifting the rice yield to a new level. The successful studies on the three-line hybrid rice and its wide application in production make a great contribution to grain production in China and other countries. However, limitation by cytoplasmic inheritance, studies on the three-line hybrid rice have failed to make great breakthroughs in recent years. Since the discovery of the dual-purpose male sterile line, the yield of hybrid rice has made a new leap forward. The successfully developed super hybrid rice and the rice growing on a trial basis have found that the two-line method has a higher advantage in rice growth and higher production-increasing potential than the three line method. Up to now, the two-line hybrid rice makes a success with the photo-thermo-sensitive male sterile line used as a genetic tool.

The photo-thermo-sensitive two-line hybrid rice needs no maintainer line, with simple seed production and low production costs. Moreover, without limit to the restorer line and maintainer line for parents of the two-line hybrid rice, the rice can crossbreed freely, thereby reducing the time for selective breeding and increasing the probability of selecting high-quality combinations. The two-line hybrid rice in China has begun to be put into production and a large quantity of experiments and production demonstrations prove that the two-line hybrid rice can increase the yield by 5% to 10% over that of the three-line hybrid rice with the same mature period. Therefore, it is a promising hybrid rice. The two-line method features free matching, low costs for seed production, no negative effects of sterile cytoplasm and easy transfer to a new sterile line. The photo-thermo-sensitive sterile line is dual-purpose. As its fertility is controlled by the photoperiod and temperature, unstable fertility easily causes fluctuations and therefore leads to certain risks in both production of hybrid seeds and reproduction. How to effectively avoid the impacts on the fertility of the photo-thermo-sensitive genic sterile line exerted by the unusual low temperature in summer has become a major problem that must be addressed in the production of the two-line hybrid rice.

Breeding of the practical photo-thermo-sensitive genic sterile line is the premise of developing the two-line hybrid rice. The so-called practicality, on one hand, means that the infertility of photo-thermo-sensitive genic sterile line remains stable during seed production and no self-pollination or seed formation happens due to a fertility fluctuation. On the other hand, the photo-thermo-sensitive genic sterile line restores fertility during reproduction and then has a high self-pollination or seed formation rate. However, the nucleus sterile genes of the photo-thermo-sensitive male sterile line bred at home and abroad come from japonica Nongken 58S, indica Annong S-1, 5460S, Hengnong S-1, Xinguang S and Zhu 1S, which have a complex genetic background and undergo a shift in the critical sterility temperature, that is, unstable fertility inheritance. During ordinary reproduction under natural conditions, the critical sterility temperature of the photo-thermo-sensitive sterile line improves internally with the higher generations. This is because there are inter-individual differences in the critical sterility temperature of the photo-thermo-sensitive genic sterile line. If no effective measures are taken to eliminate the differences, the sterile line will lose its value due to the high critical sterility temperature caused by reproduction by self-fertilization for years. During hybrid seed production, the unstable temperature easily leads to fertility fluctuations, thereby breeding low-purity seeds and even breeding seeds that are discarded. Therefore, the sterile line is almost worthless. The problem exerts a serious impact on the prospect of the two line hybrid rice as it is an obstacle to its further promotion and application.

Furthermore, though the photo-thermo-sensitive genic sterile line with a low critical sterility temperature has no fertility fluctuation in summer, its self-fertilization results in low and unstable yield and even a failure as the temperature for fertility transition from sterile to fertile is within a narrow range of 3° C., thereby causing a big restriction on the application. The purpose of breeding for the new two-line sterile line is to breed a brand-new photo-thermo-sensitive genic sterile line that is controlled by both sunlight and temperature but is mainly dominated by sunlight. That is to say, the single factor low temperature has no effects on the transfer from sterility to fertility, no matter how low the temperature is and how long the low temperature lasts. Only under the proper sunlight and temperature is the promoter that dominates the pollen development activated, thus enabling stable transfer from sterility to fertility. Sterility is jointly controlled by two climate factors, which offers double assurance to ensure the absolute safety of seed production. The research and utilization of the regulatory genes will greatly accelerate the realization of the purpose.

### DESCRIPTION OF THE INVENTION

To overcome the abovementioned drawbacks in the prior art, the present invention provides a seed production, reproduction and breeding method for the two-line hybrid rice based on the mutant of rice OsMS4 gene. With the mutation of OsMS4 (also called Carbon Starved Anther, CSA) and the abnormal development of anther in a short day condition, the microspores cannot undergo the second mitosis, leading to complete pollen abortion, so the mutant rice shows complete male sterility and can be used as a male sterile line in hybrid breeding. As a result, the osms4 male sterile plant can be used for the breeding of the two-line hybrid rice (including japonica rice and indica type rice). Changes in light conditions help give rise to self-fertilized seeds and therefore maintain the sterile line.

The present invention provides technical solutions as below.

The present invention relates to a seed production method based on the osms4 mutant, selected from the mutagenizing seeds of japonica cultivar 9522 with ⁶⁰Co gamma ray, and the stable inheritance osms4 mutant was obtained after three generations backcrossing, which is regulated by a recessive single gene OsMS4.

The said mutagenizing with ⁶⁰Co gamma ray was finished by the Radiation Chamber of Shanghai Academy of Agricultural Sciences and the ⁶⁰Co gamma ray plays the role of mutagen that induces genetic mutation in 3,000 g of the seeds of a local japonica cultivar 9522 at a dosage of 280 Gy.

The said F2 progeny is the second-generation plant that undergoes phenotypic segregation compared with wild-type plants and that is selected out after once-a-week observations in the field during the entire process extending from vegetative growth to reproductive growth among 24 individual strains. Prior to this, after soaking and hastening germination, the mutagenized seeds were sowed in May 2001. After 30 days, a single seed was individually transplanted into a farmland and grew into the first-generation mutant, that is, F1 progeny. From three spikes of rice, each seed of a F1 progeny plant was sowed separately then the seeds were transplanted into paddy land after another 30 days. Twenty-four strains of each plant were transplanted and the F2 progeny was given a number, with phenotype and segregation ratio recorded.

The present invention relates to the mutation of the OsMS4 gene developed into with use of the said seed production method.

The said rice having the osms4 mutation is a weak-photo-sensitive, medium-maturing late japonica nucleus sterile line. It has moderate plant type and erect flag leaves with regular leaf blade. The seeds are round, the spike is half-curved, medium-size and is generally crowded with 100 to 120 grains, which is less dense than a dense-eared spike. The plant is 80-90 cm tall and the leaves are dark green while the apiculus and stigma are colorless. At the 4th-5th phase of young panicle differentiation, the average temperature higher than 30° C. and more than 14 hours of sunshine duration, the osms4 mutant becomes male fertile. On the contrary, it becomes male sterile from a fertile one at the 4th-5th phase of young ear differentiation with an average temperature lower than 28° C. and less than 13 hours of sunshine duration.

The said rice having the osms4 mutation contains 16.0% of starches and has a brown rice rate of 80.0%, a milled rice rate of 70.0% and a head rice rate of 60.0%. The length-to-width ratio is 1.8, it has a low gelatinization point and a gel consistency of 70 mm and the entire growth period of the rice in the summer seeding in Shanghai is about 140 days while the process from sowing to heading lasts for around 95 days. There are about 18 leaves on the main stem.

The present invention relates to a reproduction method based on the osms4 mutant. At the 4th-6th developmental phase of young panicles, the said rice having the osms4 mutation is able to breed seeds of 150-400 kilograms/mu if exposed to periods of daylight of 14 hours a day for 15 days at a temperature of 30-32° C.

The present invention relates to the application of the said rice osms4 mutant in production of the two-line hybrid rice.

The osms4 mutant of the present invention is produced by mutagenizing seeds of japonica cultivar 9522 that are promoted at the middle and lower reaches of Yangtze River with ⁶⁰Co gamma ray. It is a result of mutation of recessive single genes, wherein the first exon of the OsMS4 gene undergoes a base deletion and A to G transversion, leading to a change in the sequence of genes.

The present invention provides the dual-purpose rice having the OsMS4 gene mutation that is able to self-pollinate with normally developed pollen under long day conditions at a high temperature and set active seeds. These seeds can grow normally after germination, allowing the inheritance of the traits of homozygous mutant genes. Under short periods of daylight in rice cultivation season, e.g. 13 hours, the osms4 mutant shows complete male sterility and can be used as a sterile line for seed production of hybrid rice. Therefore, in the climate for rice cultivation (with temperature above 25° C. on average), the control of rice cultivation time allows the regulation of periods of daylight for reproductive growth (flowering and pollen production), thereby controlling the fertility and sterility of the osms4 plant and realizing its dual purposes. That is to say, the osms4 mutant is a male fertile line under long periods of daylight and a male sterile line under short periods of daylight, which can be used for production of hybrid rice. This simplifies the seed production of hybrid rice and addresses the problem of “shift” caused by temperature changes during seed production of thermo-sensitive sterile rice.

With free combinations of the osms4 sterile line, the probability of selecting excellent combinations is far higher than that of the three-line method, avoiding the negative effects of sterile cytoplasms on heterosis and simplification of cytoplasms. In addition, it is proved that when the mutation sequence is induced to other types of rice of different genetic backgrounds after the crossbreeding of osms4 and other types of rice, these plants containing osms4 homozygote will also become fertile under long periods of daylight and sterile under short periods of daylight, which can be used for production of the two line hybrid rice. Therefore, the OsMS4 locus is one of the molecular markers that can be widely used in cultivation of the new two-line hybrid rice and has broad application prospects in pyramiding using molecular markers.

### BRIEF DESCRIPTION OF THE DRAWINGS

FIG. 1 shows the views of the present invention;

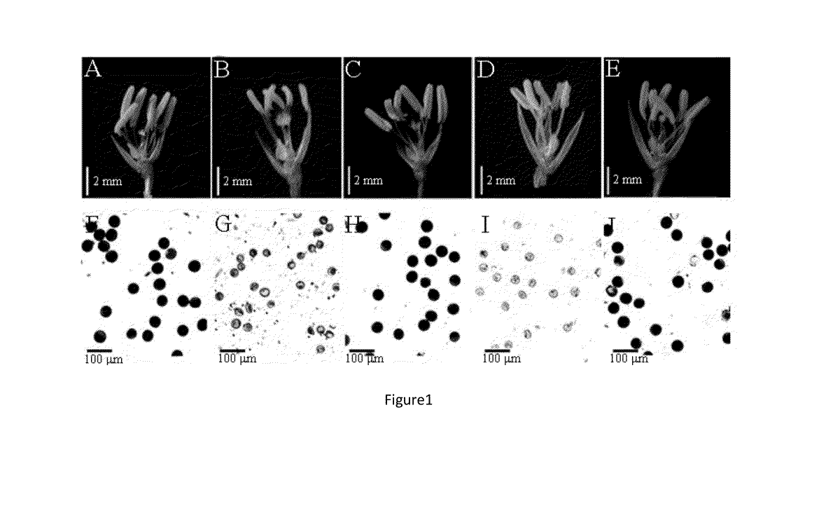

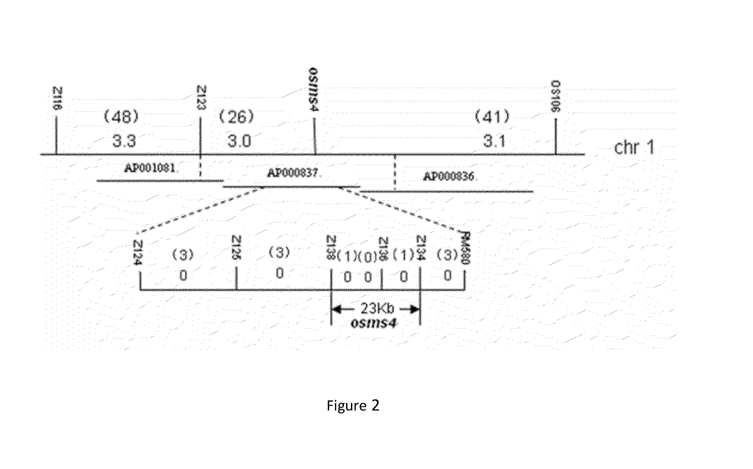

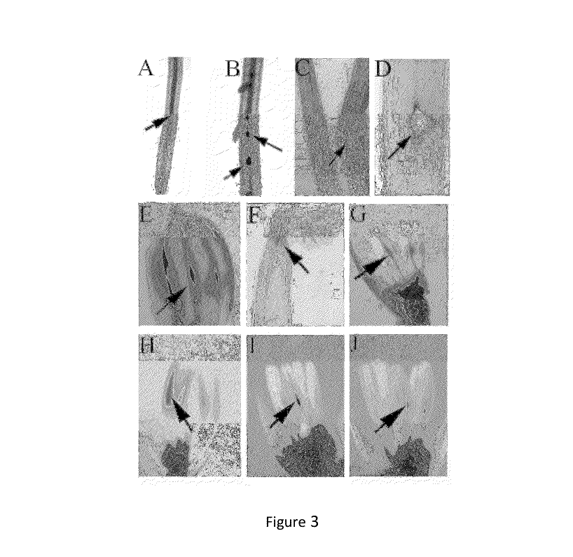

wherein FIG. 1A shows the view of japonica cultivar 9522 with palea and lemma removed; FIG. 1B shows the view of osms4 sterile line with palea and lemma removed; FIG. 1C shows the view of the osms4 sterile line undergoing transformation of OsMS4 genomic DNA expression vector with palea and lemma removed; FIG. 1D shows the view of japonica cultivar 9522 undergoing transformation of OsMS4 RNAi expression vector with palea and lemma removed; FIG. 1E shows the view of the osms4 photo-thermo-sensitive sterile rice with palea and lemma removed; FIG. 1F shows the view of staining of the I2-IK pollen grain from japonica cultivar 9522; FIG. 1G shows the view of staining of the I2-IK pollen grain from the osms4 sterile line; FIG. 1H shows the view of staining of the I2-IK pollen grain from the osms4 sterile line undergoing transformation of OsMS4 genomic DNA expression vector; FIG. 1I shows the view of staining of the I2-IK pollen grain from japonica cultivar 9522 undergoing transformation of OsMS4 RNAi expression vector and FIG. 1J shows the view of staining of the I2-IK pollen grain from OsMS4 photo-thermo-sensitive sterile rice. Icons in FIG. 1A to 1E refer to 2 mm while those in FIG. 1F to 1J refer to 100 microns.

FIG. 2 shows the mapping of the OsMS4 gene locus on the chromosome.

Numbers marked on the vertical bar refer to the names of primers used; those starting with “AP” are names of BAC clones; a centimorgan (cM) is a unit for measuring genetic distance; numbers in parentheses represent the recombination events in mapping populations.

FIG. 3 shows the tissue specificity of GUS genes driven by the OsMS4 promoter.

FIGS. 3A and 3B show the root; FIG. 3C shows the blade; FIG. 3D shows the injured blade; FIG. 3E shows the husk; FIG. 3F shows the pistil; and FIG. 3G to 3J show the anther at different stages.

### DETAILED DESCRIPTION

The embodiments of the present invention are further detailed as below and are carried out on the premise of the technical solutions provided by the present invention with specific methods and procedures. However, the present invention is not limited to the embodiments below.

### Embodiment 1

Getting the Improved Mutant (osms4) Plant

The mutant is produced by mutagenizing the seeds of japonica cultivar 9522 at a dosage of 280Gy with the ⁶⁰Co gamma ray, backcrossing one male sterile mutant of mutagenized F2 progeny for three generations, obtaining the rice plants having the OsMS4 gene mutant with stable inheritance which are regulated by a recessive single gene. All the plants are planted at the test site of Shanghai Academy of Agricultural Sciences. The OsMS4 gene mutant backcrosses with japonica cultivar 9522, producing the F1 progenies that are all fertile. The separation takes place in the F2 progenies, forming 419 normal plants and 126 mutant plants with the ratio close to 3:1 (χ2=1.028, P>0.05), which demonstrates that the phenotype of the male sterile mutant results from the mutation of a single nuclear gene.

### Embodiment 2

Studying the Relationships Between Fertility of the Improved Mutant (the osms4 Mutant) and Thermo-Photoperiod Conditions

Many years of experiments and studies have found that under the natural conditions the young ear differentiation stage is a period when the metrocytes of pollen take shape. Exposed to a less than 13 hours of daylight per day, the mutant shows stable sterility and has white anthers and typical abortive pollen. Under more than 14 hours of daylight per day at a temperature ranging from 25° C. to 35° C., the mutant can become fertile from sterile, with normal pollen staining, a pollen grain producing rate of 45-80% and a seed setting percentage of 60-80%. The transfer of fertility is sensitive to sunlight duration, sunlight intensity and spectral range. If the sunlight duration falls short of the requirement for fertility transfer, it will be insensitive to the fluctuations in the temperature.

At the 4th-6th developmental phase of young panicles, the photo-thermo-sensitive genic sterile line is able to breed sterile lines of around 150-400 kilograms/mu if exposed to 14 hours of daylight a day for 15 days at a daily average temperature of 30-32° C.

### Embodiment 3

Mapping Mutational Locus to Obtain Improved Genes

(1) Mapping population. It refers to the male sterile line chosen from the F2 progeny that is obtained by self-pollination after osms4 crossbreeds with indica type rice Longtefu B.

(2) Rice DNA extraction. The DNA extraction is done by the perfected CTAB method. Specifically, 0.1-0.2 gram of blade (about a half blade) is placed into a mortar and ground immediately with the right amount of liquid nitrogen. The powders are put into a 2 ml centrifuge tube, mixed with 700 μL of 1.5×CTAB solution preheat at 100° C., placed in a 56° C. water bath for 20 minutes and then taken out of the centrifuge tube. The mixture is blended with the equal amount of chloroform/isoamyl alcohol. After centrifugation at 13,000 rpm for 10 minutes, the supernatant fluid is transferred to a new tube and placed for more than half an hour at 20° C. below zero after being blended with 900 μL of absolute ethyl alcohol. The DNA separated out is centrifuged at 14,000 rpm for 10 minutes. With supernatant fluid removed, the precipitates are washed once with 1 ml of 70% ethanol, centrifuged and dried. The precipitates dissolve into 200 μL of 1/10 TE or water and are stored at a 4° C. freezer.

(3) InDel molecular marker analysis. Based on the comparison to the nucleotide sequences of japonica Nipponbare and indica type rice 9311 that are released, to design primers for the differences and testify to the polymorphism between two parents, namely japonica 9522 and indica type rice Longtefu B. The order for PCR amplification is 1 μL template, 1 μL 10 pmol/μL Primer 1, 1 μL 10 pmol/μL Primer2, 1 μL 10× Buffer (Mg2+), 1 μL 2 mM dNTP, 0.1 μL Taq and 3.9 μL of water in the 10 μL system. The PCR products are detected by 6% polyacrylamide gel electrophoresis (PAGE) and silver staining.

(4) Bulked segregant analysis. Amplification reactions are performed with 132 pairs of markers. The results indicate that the ZH104 marker on the No. 1 chromosome interlocks with the OsMS4. The marker ZH106 nearby is used to further test among the separated populations from the F2 progenies. The results of the test show that the marker is also linked with the OsMS4 which is preliminarily mapped between ZH104 and ZH106. To further map the OsMS4, the F2 progenies are expanded to 3,000 plants, among which 750 plants are mutants. Among the eight InDel markers (as shown in Table 1), the OsMS4 is finally mapped between ZH134 and ZH138. According to the splice junction sequence of the No. 1 chromosome of japonica Nipponbare downloaded from a website (http://www.tigr.org), the marker ZH134 and ZH138 locate on the clone AP00837 with a physical distance of 23 kb (FIG. 2). The genotyping is conducted on the single male sterile plant in the mapping populations with the molecular markers. The MapDraw V2.1 is used to draw a linkage map for molecular markers in the targeted gene regions.

TABLE 1
InDel molecular markers
and their nucleotide sequence

| Molecular markers | Sequence of upstream primers | |
|---|---|---|
| ZH116-3 | 5′ CAAGCTGTGGTCGCGTTCCT3′ | |
| (SEQ ID NO: 14) | | |
| ZH124 | 5′ TTGCTTTCGCCTTAATTATTT 3′ | |
| (SEQ ID NO: 15) | | |
| ZH125 | 5′ CAATGGTGTAAGGAAGGATG3′ | |
| (SEQ ID NO: 16) | | |
| ZH127 | 5′ AAGCTTGGCCTGATGGGA 3′ | |
| (SEQ ID NO: 17) | | |
| ZH128 | 5′ GCGACTTTGCACGTAGATGT 3′ | |
| (SEQ ID NO: 18) | | |
| ZH131 | 5′CAGAAATGCATAAATGCCACA3′ | |
| (SEQ ID NO: 19) | | |
| ZH134 | 5′ GGGGGGTGAATAGGCTTTC 3′ | |
| (SEQ ID NO: 20) | | |
| ZH138 | 5′TAAATACTTGTGTGCATGGATG3′ | |
| (SEQ ID NO: 21) | | |
| | Sequence of | |
| Molecular markers | downstream primers | Types |
| ZH116-3 | 5′AGGGCGAGTAAGGGAACAACG3′ | InDel |
| (SEQ ID NO: 22) | | |
| ZH124 | 5′ CTGCCTACGGCTATGTGCT 3′ | InDel |
| (SEQ ID NO: 23) | | |
| ZH125 | 5′GTTTGGTTGGTTGTGAGGG3′ | InDel |
| (SEQ ID NO: 24) | | |
| ZH127 | 5′ TGGTGTATTTTATCGGGGTTG 3′ | InDel |
| (SEQ ID NO: 25) | | |
| ZH128 | 5′ ATGATCAATAGTTGCTCCCTTT 3′ | InDel |
| (SEQ ID NO: 26) | | |
| ZH131 | 5′ GAAGAACCACCCTAACCAAA 3′ | InDel |
| (SEQ ID NO: 27) | | |
| ZH134 | 5′GTGTCTGCTAGGGTTACAGGTTT3′ | InDel |
| (SEQ ID NO: 28) | | |
| ZH138 | 5′ TTATTTTTCACTCGTCCCAC 3′ | InDel |
| (SEQ ID NO: 29) | | |

(5) Clone of OsMS4 genes. There are two foreseen genes within the scope of 23 kb, wherein one is an MYB gene. The DNA sequencing of a mutant and a wild-type gene finds that the first exon of the MYB gene undergoes a nucleotide deletion and G to A transversion. The mutated gene is named OsMS4.

The sequence of complementary DNA (cDNA) of the said OsMS4 gene is SEQ ID NO. 1 and the coding sequence (CDS) of the OsMS4 gene is SEQ ID NO. 2. The sequence for its genome is SEQ ID NO. 3 while a protein of the OsMS4 gene has the amino acid sequence shown in SEQ ID NO. 4. The CDS of the mutated OsMS4 gene is SEQ ID NO. 5.

### Embodiment 4

Genetic Engineering Technology Recovers Fertility of osms4 Sterile Line

With genome's DNA of japonica cultivar 9522 as a template and MYBF (SEQ ID NO.6) and MYBR (SEQ ID NO.7) as primers, a 4318 bp fragment is amplified, including the full length, promoter and terminator of the OsMS4 gene. The PCR has a total volume of 50 μL, including 1 μL (200 ng) of rice genome's DNA template, 5 μL 10×KOD enzyme reaction buffer, 3 μL 25 mM MgSO4, 5 μL 2 mM dNTPs, 1.5 μL 10 μM primers respectively, 1 KOD enzyme (TOYOBO) and a certain amount of ddH2O (added to form 50 μL of PCR). The reaction procedures include denaturation at 94° C. for 5 minutes, at 94° C. for 40 seconds, at 55° C. for 40 seconds and at 68° C. for 4.5 minutes, 35 cycles and the extension at 68° C. for 5 minutes. The PCR products are detected on the 1% agarose gel and undergo tapping for purification. The purified fragments are directly digested with PmaCI and BamHI and linked to the pCAMBIA 1301 vector after the same enzyme digestion, the sequencing is proved to be correct and the vector is used to transform EHA105 agrobacterium and to the 9522 rice by agrobacterium-mediated transformation (Hiei, et al., efficient transformation of rice (Oryza sativa L.) mediated by agrobacterium and sequence analysis of the boundaries of the T-DNA. Plant Journal 1994, 6: 271-282). As shown in FIG. 1, after the complementary vector is transformed to the osms4 mutant, the anthers become yellow and I2-IK stainable pollen grains restore staining, thereby generating normal seeds. The results demonstrate that OsMS4 gene can restore fertility of osms4 mutant.

### Embodiment 5

RNA Interference (RNAi) Transforms and Tests Gene Function

RNAi fragments are designed on the last exon of the OsMS4 gene and 3-UTR untranslated region (3-UTR). Primers, namely, OsMS4i-1F (SEQ ID NO.8), OsMS4i-1R (SEQ ID NO.9), OsMS4i-2F (SEQ ID NO.10) and OsMS4i-2R (SEQ ID NO.11), are designed. The PCR has a total volume of 20 μL, including 1 μL (about 50 ng) rice genome's DNA template, 1× Taq enzyme reaction buffer, 1.2 μL 25 mM MgCl2, 1.5 μL 2 mM dNTP, 0.2 μL 10 μM various primers respectively, 2 μL 50% glycerol, 0.3 Taq enzyme (Takara) and a certain amount of ddH2O (added to form 20 μL of PCR). The reaction procedures include denaturation at 94° C. for 5 minutes, at 94° C. for 40 seconds, at 55° C. for 40 seconds and at 72° C. for 40 seconds, 35 cycles and the extension at 72° C. for 5 minutes. The PCR products are detected on the 1.5% agarose gel and undergo tapping for purification. Two purified fragments are linked to the pTCK303 RNAi vector respectively. The vector is used to transform EHA105 agrobacterium and to the 9522 rice by agrobacterium-mediated transformation (Hiei, et al., efficient transformation of rice (Oryza sativa L.) mediated by agrobacterium and sequence analysis of the boundaries of the T-DNA. Plant Journal 1994, 6: 271-282). In the RNAi transgenic plant, the anthers are white and pollen grains are unstained (as shown in FIG. 1). This indicates that the OsMS4 gene is capable of controlling the fertility of rice.

### Embodiment 6

OsMS4 Promoter Initiates the Tissue Specific Expression of GUS Reporter Genes

With primers of MYBF (SEQ ID NO.6) and OsMS4-Promoter R (SEQ ID NO.12) and wild-type genome DNA as template, the PCR results in the promoter sequence (SEQ ID NO.13). The PCR has a total volume of 50 μL, including 1 μL (about 200 ng) rice genome's DNA template, 5 μL 10×KOD enzyme reaction buffer, 3 μL 25 mM MgSO₄, 5 μL 2 mM dNTPs, 1.5 μL 10 μM various primers respectively, 1 KOD enzyme (TOYOBO) and a certain amount of ddH₂O (added to form 50 μL of PCR). The reaction procedures include denaturation at 94° C. for 5 minutes, at 94° C. for 40 seconds, at 55° C. for 40 seconds and at 68° C. for 2.5 minutes, 35 cycles and the extension at 68° C. for 5 minutes. The PCR products are detected on the 1% agarose gel and undergo tapping for purification. The purified DNA is added with a tail by Taq enzyme (Takara), including 5 μL 10× Taq enzyme reaction buffer, 5 μL 2 mM dNTPs and a certain amount of water (added to form 50 μL of Taq enzyme). The reaction procedures include the extension at 72° C. for 5 minutes. The products are detected on the 1% agarose gel and undergo tapping for purification. The purified DNA is linked to the pMD18-T (Takara) and digested with BamHI and NcoI. The fragment is recycled and linked to the pCAMBIA1301 vector that undergoes the same enzyme digestion, obtaining the corresponding clones of the pCAMBIA1301, promoter plus GUS genes. The vector is used to transform EHA105 agrobacterium and to the 9522 rice by agrobacterium-mediated transformation (Hiei, et al., efficient transformation of rice (Oryza sativa L.) mediated by agrobacterium and sequence analysis of the boundaries of the T-DNA. Plant Journal 1994, 6: 271-282). Following the production of a transgenic plant, the tissue staining is performed (see Jeferenson, R A (1987) Plant Mol Biol Rep).

As shown in FIG. 3, GUS genes show specific expression of the vascular bundle in the root, blade ring, injured tissue, vascular bundle in the husk, pistil and vascular bundle in the anthers at different developmental phases.

### Embodiment 7

Seed Production by Crossbreeding the osms4 Photo-Thermo-Sensitive Male Sterile Line with the Restorer Line

The seeding in Shanghai in the autumn of 2008 covered an area of 1.0 mu. The male parent, which was the photosensitive big-spike japonica restorer line JP50, was sowed on May 30. Rice seedlings were transplanted on June 25 and the rice eared on September 5. The female parent was sowed on June 23, with transplanting of rice seedlings on July 12 and earing on September 8. The male and female parents were at a spacing ratio of 2:8. The female parent was sampled at the first day of flowering to receive a microscopy. The typical abortion was found in 80% of pollens stained with I2-IK and spherical pollen abortion in 20% of pollens. The self-fed seed setting rate after bagging stood at zero, the female parent bloomed early with excellent overlapping of flowering of the male and female parents and there were 140 kg of hybrid seeds set. It was found that the sterile plant rate reached 0.5% and the purity was 97.8% upon purity identification in Hainan in the winter of 2008.

The seeding in Hainan in the winter of 2008 covered an area of 1.5 mu. The male parent, the thermo-sensitive restorer line JP69, was sowed on December 10. Rice eared on March 1. The female parent was sowed on December 31, with earing on March 4. The osms4 female parent had pure white short rod-like anthers and set no seeds upon self-pollination after bagging. It bloomed late and saw a decrease in seed production upon crossbreeding, setting 30 kg of seeds per mu, under short periods of daylight, low humidity conditions and at a low temperature. It was found that the purity was 96.7% and the sterile plant rate reached 1% upon purity identification for the first-generation hybrid rice in Shanghai in the normal season of 2009.

### Embodiment 8

Free Combinations of osms4 Photo-Thermo-Sensitive Male Sterile Line

With free combinations of the osms4 sterile line, the probability of selecting excellent combinations is far higher than that of the three line method, avoiding the negative effects of sterile cytoplasms on heterosis and simplification of cytoplasms. The osms4 sterile line has undergone a test cross and grouping with the restorer line and conventional rice of different ecotypes since 2006. The crossbreeding of the photo-thermo-sensitive sterile line with the early-maturing C418 japonica restorer line produces the first-generation hybrids that feature early maturity, big spikes, high resistance and growth vigor. The combination of the photo-thermo-sensitive sterile line with big-spike japonica restorer lines JP69 and JP50 produces the first-generation hybrids that feature sturdy stalks, big spikes, dense grains, high lodging resistance and high seed setting rate and have agronomic traits and technical indicators of the super hybrid rice. The crossbreeding of the photo-thermo-sensitive sterile line with conventional Xiushui and Guihuahuang japonica rice gives rise to the first-generation hybrids that show the characteristics of well-shaped leaves, strong tillering ability, moderate maturity, high seed setting rate, good yield stability and easiness to cultivate. Meanwhile, the sterile line is crossed with 9311 indica type rice and Longtefu rice, giving rise to the first-generation hybrids that show the characteristics of growth vigor, well-shaped leaves and a seed setting rate of 50% to 70%. These demonstrate that the sterile line shows wide compatibility.

### Embodiment 9

Inheritance, Fertility Transfer and Application of OsMS4 Gene Locus

When the mutation sequence is induced to other types of rice of different genetic backgrounds after the crossbreeding of the osms4 and other types of rice, these plants containing the osms4 homozygote will also become fertile under long periods of daylight and sterile under short periods of daylight, which can be used for production of the two line hybrid rice. The osms4 line was crossed with Zhenshan 97B, Tianfeng B, No. 1 Feng'aizhan and early-maturing japonica Songjiang Xiangjing in 2007, producing F2 progenies that conform to the separation law of 3:1 through bulked segregant analysis. The molecular marker analysis was used to backcross the sterile plants having the osms4 genes with cross parents. The B1F2 progenies showed stable sterility in Hainan while the single sterile plant with rice root was taken back to Shanghai for re-growth and reproduction, with its fertility observed under different sunlight and temperature conditions. It was found that ⅓ of plants showed characteristics of fertility alteration of OsMS4, suggesting that these plants have a fertility restoring and seed setting rate of 60%. PCR assays are performed on samples of these plants, which confirmed that there are OsMS4 molecular markers (SEQ ID NO.5). Seeds set were taken to Hainan and sowed by stages and it was found that plants were sterile during earing at different stages. Abortive pollens were found upon a microscopy for anthers and the typical abortion set no seeds upon self-pollination after bagging. The test results indicated that as Hainan (the latitude and longitude of Sanya is 18° 14′ N/109° 30′ E) is near the equator, it generally receives direct solar radiation and experiences short periods of daylight (shorter than 13 hours from January to March), so the sunlight duration falls short of the requirement for sterility gene transfer. As a result, the seed production in Hainan is safe and reliable.

What is claimed is:

1. A method for generating an osms4 mutant rice seed, characterized in that, comprising one male sterile mutant selected from the mutagenizing seeds of japonica cultivar 9522 with ⁶⁰Co gamma ray, and the stable inheritance of said osms4 mutant is obtained after three generations of backcrossing, which is regulated by a recessive single gene OsMS4.

2. The method for generating an osms4 mutant rice seed according to claim 1, characterized in that the mutagenizing is performed with the ⁶⁰Co gamma ray at a dosage of 280Gy.

3. The method for generating an osms4 mutant rice seed according to claim 1, said osms4 mutant comprises a mutant nucleic acid having the nucleic acid sequence of SEQ ID NO: 1.

4. The method for generating an osms4 mutant rice seed according to claim 1, said osms4 mutant is able to express a mutated protein having amino acid sequence of SEQ ID NO: 4.

5. The method for generating an osms4 mutant rice seed according to claim 1, characterized in that at the 4^{th}-6^{th} developmental phase of young panicles (during the formation of meiosis and pollen grain development), said osms4 mutant is able to produce normal pollen grains and self-fertilize when exposed to more than 13.5 hours of daylight a day for more than 15 days with a temperature more than 25° C.

6. An isolated nucleic acid having the nucleic acid sequence of SEQ ID NO: 13.

7. The isolated nucleic acid according to claim 6, said nucleic acid is able to be ligated with a target gene which is utilized to make a transgenic plant, said target gene can express in a specific tissue in the plant.

8. The isolated nucleic acid according to claim 7, said specific tissue is a vascular bundle in the root, a blade ring, an injured tissue, a vascular bundle in the husk, a pistil and a vascular bundle in the anthers at different developmental phases.

9. An isolated nucleic acid having the nucleic acid sequence being homology with SEQ ID NO.13 by 70% or more.

10. The isolated nucleic acid according to claim 9, said nucleic acid is able to be ligated with a target gene which is utilized to make a transgenic plant, said target gene can express in a specific tissue in the plant.

11. The isolated nucleic acid according to claim 10, said specific tissue is a vascular bundle in the root, a blade ring, an injured tissue, a vascular bundle in the husk, a pistil and a vascular bundle in the anthers at different developmental phases.

12. A vector comprising a nucleic acid having the nucleic acid sequence of SEQ ID NO.13.

13. The isolated nucleic acid according to claim 12, said vector is able to be inserted by a target gene which is utilized to make a transgenic plant, said target gene can express in a specific tissue in the plant.

14. The isolated nucleic acid according to claim 13, said specific tissue is a vascular bundle in the root, a blade ring, an injured tissue, a vascular bundle in the husk, a pistil and a vascular bundle in the anthers at different developmental phases.

15. A peptide having the amino acid sequence of SEQ ID NO: 4.

16. The peptide according to claim 15, a nucleic acid being able to encode said peptide can be utilized to make a transgenic plant.
